(19) United States
(12) Patent Application Publication
(10) Pub. No.: US 2003/0144625 A1
(43) Pub. Date: Jul. 31, 2003

(54) Method and apparatus for the treatment of infections of the nail matrix and nail plate

(76) Inventors: Garry Sherman, Basking Ridge, NJ (US); Bernard Covalesky, Randolph, NJ (US); Scott Karolchyk, Newton, NJ (US)

Correspondence Address: DUANE MORRIS LLP, 100 COLLEGE ROAD WEST, SUITE 100, PRINCETON, NJ, 08540-6604, US

(21) Appl. No.: 10/345,865

(22) Filed: Jan. 16, 2003

Related U.S. Application Data
(60) Provisional application No. 60/352,490, filed on Jan. 28, 2002.

(51) Int. Cl. A61N 1/30

(52) U.S. Cl. 604/20; 604/289

(57) ABSTRACT

There is disclosed a method and protocol for the treatment of various infections of the nail matrix and nail plate. The technique uses iontophoresis. Using iontophoresis, one specifically applies an electrode which overlies the skin of the nail matrix and nail plate itself. The method uses various disease specific chemicals that are normally used to treat, cure and provide prophylaxis to various infective micro-organisms, which include fungal, yeast, mold and other antimicrobial agents. The chemicals and medications are prepared in the form of an ionized solution. The ionized solution is used to saturate a conductive pad. The conductive pad, as indicated, is placed over the infected nail area, including the nail matrix and forms a first electrode. A second electrode is placed in close proximity on another part of the patient's body to cause a generator which is connected to both electrodes to generate a current of a relatively small value. The current causes the medication to be transported into the nail matrix. The invention can achieve markedly better cure rates than present methods of orally administrating the drugs without any adverse systemic side effects.

### RELATED APPLICATIONS

This application claims priority based on a provisional application filed for the inventors entitled, “A Method and Protocol for the Treatment, Cure and Prophylaxis of Various Infections of the Nail Matrix and Nail Plate”. The provisional application is Application No. 60/352,490 filed on Jan. 28, 2002 for the inventors herein. Priority is claimed as of that date.

### FIELD OF INVENTION

This invention relates to a method and apparatus for the treatment of various infections of the nail matrix and nail plate, and more particularly, to a method and apparatus for the treatment of fungal infections of the nail.

### BACKGROUND OF THE INVENTION

As one can ascertain, the present recommended treatment for Onychomycosis (a fungal infection of the nails) is oral antifungal medication. See, for example, an article entitled, “Management of Toenail Onychomycosis” by Tom C M, Kane M P, published in the American Society of Health Systems Pharmacists, May 1999, volume 56, pages 865 to 871. See also a publication entitled, “Onychomycosis in Diabetes, Management Considerations” by Albreski, Gupta and Gross, published in the Podiatric Dermatology Clinic, Veterans Affairs Connecticut Healthcare System, 555 Willard Avenue, Newington, Conn. 06111. This routine treatment has numerous serious side effects. These side effects can result in a severe disturbance of liver function, which is particularly dangerous for elderly, diabetic and other people with compromised immune systems. When these drugs are also administered transdermally in the form of creams and ointments in the treatment of the nail fungal infections, their systemic side effects are eliminated, but their effectiveness in curing the condition is substantially reduced.

Apart from the adverse effects of such medications, there are further problems in regard to presently used medications. Based on recent studies, 15 to 20 percent of people between the ages of 40 to 60 have Onychomycosis. In the elderly, the percentage of this infection increases to greater than 30 percent. One third of known diabetics have a fungal infection, which has been proven to increase the risk of life threatening infections of the foot. According to Medicare reports, in a single year, patients over 65 years of age have recorded some 1,300,000 Onychomycosis-related visits which cost more than 43 million dollars in healthcare expenses. In addition, the cost of fungal infection in diabetic patients may result in amputation. It is estimated that by the year 2025, the diabetic population will increase to 300 million people in the world. One drug that has been utilized orally is sold under the trademark Lamisil. The drug, as indicated, is probably one of the most effective of the oral medications currently prescribed for the treatment of Onychomycosis and requires a patient to take 250 milligrams a day for three months, totaling 21,000 milligrams.

In any event, the following side effects can be ascertained and are listed in the PDR, which is the Physicians' Directory Reference. These include gastrointestinal symptoms, such as diarrhea, dyspepsia, abdominal pain, nausea, flatulence and dermatological symptoms such as rash, pruritis, urticaria. Other problems involve liver enzyme abnormalities, taste disturbances, visual disturbances, symptomatic idiosyncratic hepatobiliary dysfunction (including cholestatic hepatitis), serious skin reactions, allergic reactions, changes in the ocular lens, decreases in the lymphocyte count and generally many other serious symptoms, including hair loss, malaise and fatigue.

There is a distinct lack of studies on drug interactions in regard to the use of this drug with others. It is very well understood that oral administrations of antifungal drugs are delivered via the vascular system and therefore, they achieve better penetration than current topical regimes of treatment. As effective as the drugs are, the basic cure rate is limited. Topical treatment of Onychomycosis has only had partial success because the pathogens ultimately invade the nail matrix as well as the nail plate. Both the matrix and the nail plate are difficult to penetrate by normal skin vehicles, which are, for example, creams, oils and so on.

It is therefore an object of the present invention to alleviate the above-noted problems associated with oral and topical antifungal medication and provide an apparatus and method which is safer and more effective.

### SUMMARY OF INVENTION

The present invention utilizes the process known as Iontophoresis. Iontophoresis is utilized as follows. A source of current is obtained from a battery, the battery conventionally has a positive and negative electrode. One electrode is placed on one portion of a person's limb, such as, for example, the lower leg of a person when a person's toenails are being treated. The other electrode is placed over the nail plate and the nail matrix. This electrode may include an effective amount of any one or combination of the following antifungal, antimicrobial medications: Terbinefine Hydrochloride, Griseofulvin, Potassium Permanganate, Ketocanazole, Clotrimazole, Cicloprox Olamine, Tolnaftate, Vinegar, Copper Sulfate, Econazole Nitrate, Tioconazole, Undecylenic Acid, Proprionic Acid, Imadazole, Nystatin, etc. The electrode which is placed over the nail may be a combination of gauze, plastic and other material which is capable of absorbing and holding an ionized solution of the above noted effective medication. The battery or generator provides a low amperage current, which current is directed from the negative electrode to the positive electrode to cause the medication which is saturated on one of the electrode pads to diffuse into the nail matrix. The medication is actually driven into the nail matrix by the generated charge. This procedure is effective and one can utilize relatively small doses of the above-noted drugs over a period of weeks. The cure rates equal or better than those which utilize oral administration of medicines and have absolutely no serious side effects.

### BRIEF DESCRIPTION OF THE FIGURES

FIG. 1 depicts a diagram showing the application of the method according to this invention being employed upon the foot of a patient where a positive/negative electrode are connected to a generator (a battery) and where medication is forced into the infected nail and nail matrix by the process known as Iontophoresis.

FIG. 2 depicts an alternate embodiment of an electrode configuration which can be employed in accordance with this invention.

FIG. 3 is another alternate embodiment of an electrode which can also be employed according to the teachings of this invention.

FIG. 4 depicts a flow chart of the method employed according to this invention.

### DETAILED DESCRIPTION OF THE FIGURES

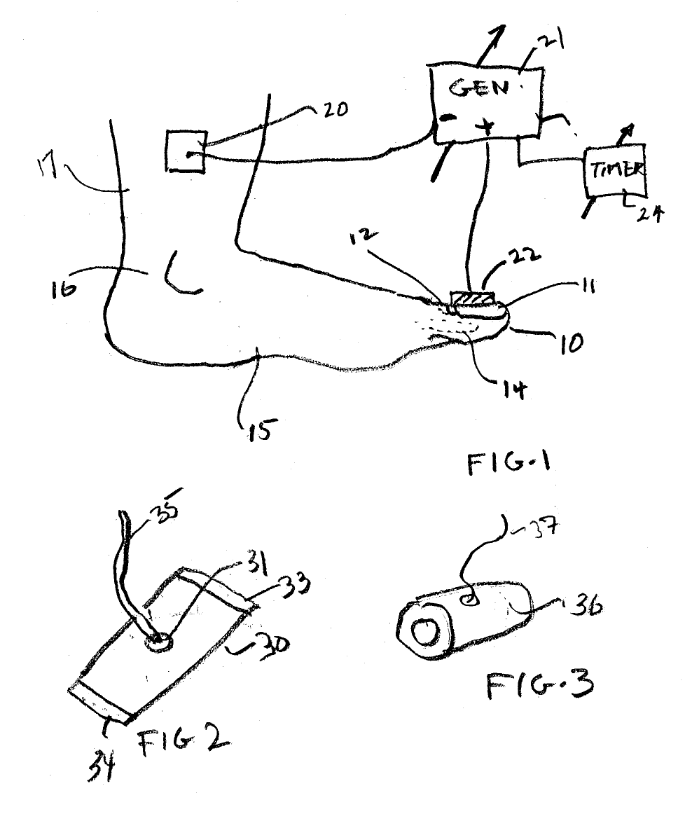

Shown in FIG. 1 is a replica of a foot 15 of a patient who has, for example, a nail infection which infects the big toenail 11 of the patient. It is, of course, understood that any other toe or fingernail can be treated accordingly. In regard to FIG. 1, numeral 11 references the toenail and numeral 12 is indicative of the location of the nail bed of the patient. There is shown the distal phalanx 14 in a dashed line representation. The distal phalanx, of course, is the bone which is associated with the large toe. Also seen is the patient's foot designated by reference numeral 15, the ankle 16 and the portion of the lower leg 17. It is seen that on a portion of the lower leg right above the ankle, there is placed or positioned a negative electrode pad 20. The negative electrode pad 20 is directed to the negative terminal of a generator 21 which may, for example, be a battery or other current generating device. The positive electrode of the battery is connected to a positive electrode pad 22. The positive electrode pad, as will be explained, is saturated or otherwise treated with an ionized solution of medication, which is effective for treating a fungal disease. This medication essentially will be driven into the nail matrix by use of the current flowing through the electrodes via generator 21. There is also shown a timer 24, which is connected to the generator 21. The timer 24 may be an ordinary timing device which is available in many different embodiments. The timer may be set for a predetermined treatment period, while the practitioner sets the generator for a desired current flow. The timer will end the session and therefore, shut off the generator 21 after the predetermined treatment period. The generator 21 is a low amperage generator capable of producing a current between one to five milliamps. The electrode 22 may be made of the combination of gauze, plastic or other material which is capable of absorbing and holding the solution containing the medication. The electrode 22 is also conductive, as is understood. The current flows between the electrode pad terminals 20 and 22, through the generator. As one will ascertain, the impedance between a portion of the foot, as defined by electrode 20, and between the toe 22, is a relatively high impedance. A current will flow, and the current flow is maintained through the body. The process of causing current flow by the technique of Iontophoresis is extremely well known. The method and protocol using this phenomenon is about 200 years old. Iontophoresis enables various chemicals and medications to penetrate the skin by driving the ions of these substances into the intracellular layers of the skin with either small negative or small positive electrical charges. There are many devices available today that are capable of transdermal molecular delivery, which essentially enables one to use the Iontophoresis method.

By using Iontophoresis and by applying it specifically to the overlying skin of the nail matrix and the nail plate itself, provides a new and unexpected result. Namely, one by the use of various disease specific chemicals and medications can treat, cure or provide prophylaxis of various infective microorganisms (fungal, yeast, mold and other antimicrobial agents) of the nail and nail matrix. The treatment employs specifically prepared medications known to be effective against these specific microorganisms. The chemicals and medications are prepared in the form of an ionized solution, which is then delivered directly into the infected areas of the nail and the nail matrix. The nail matrix 12 as shown in FIG. 1 lies beneath the skin, posterior to the nail. This area is extremely difficult to penetrate by other techniques and that is why, as indicated above, the presently used treatment of choice relies on oral antifungal medications, which penetrate the nail matrix via the bloodstream. Unfortunately, large amounts of these drugs must be given orally. For example, Lamisil calls for 21,000 milligrams over a period of three months, with the above-noted potential serious side effects. The present method and protocol enables one to utilize a very small amount of the chemical. For instance, in the treatment of a nail disease caused by Tinea Rubrum and Mentagrophytes, this invention can enable one to use several medications at a fraction of the oral dosage. Particularly in utilizing the medication, Lamisil, 1/6000^{th} of its normal oral dosage is used. Furthermore, Lamisil, because it is not orally ingested, never passes through the digestive system, thereby completely avoids the above-noted systemic problems. It is also apparent that the apparatus and method, as described, can achieve markedly higher cure rates than the oral method of administration of these anti-fungal drugs. This improved cure rate occurs without any systemic side effects and only minimal local side effects. This is accomplished by bypassing the systemic route used by oral medication and placing the medication exactly where it is needed.

The Iontophoretic approach of the treatment of infected nails and the nail matrix requires the combining of several different techniques which are utilized specifically for the cure and treatment and prophylaxis of diseased nails of the fingers and toes. FIG. 2 shows another electrode configuration 30. A center portion 31 is conductive and is surrounded by a mesh or gauze 32. The member 30 can receive an ionized solution of medication and has Velcro or adhesive ends 33 and 34 to position the electrode about a finger or toenail. The wire 35 is connected to the proper terminal of the generator 21 (+ or −).

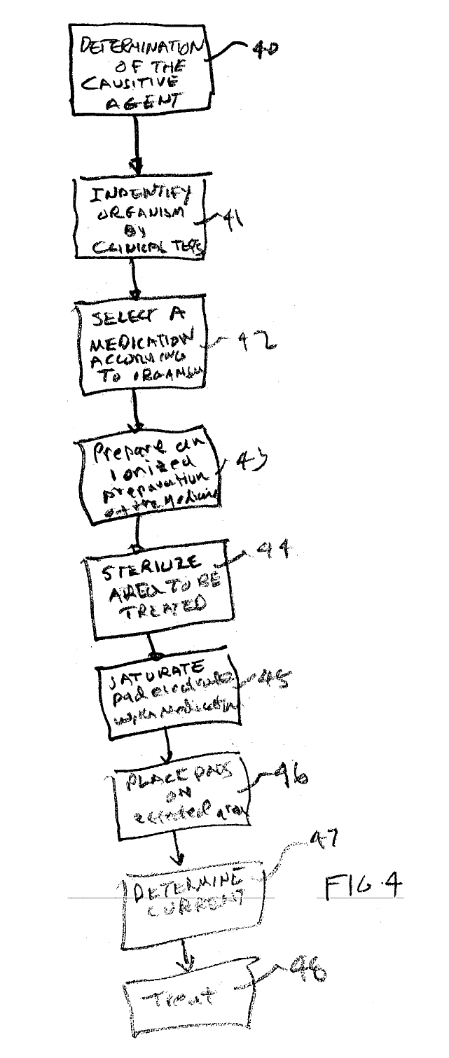

FIG. 3 shows another electrode configuration 36 which can be used to insert a toe or finger. The electrode is cylindrical and one can place a toe or finger into the cylinder. The electrode can be saturated with an ionized solution, as the cylinder is fabricated from a sponge material or other absorbent material. A wire 37 is connected to a conductive layer associated with the electrode configuration 36.

Referring to FIG. 4 there is shown a flow chart of a treatment procedure according to this invention. Reference numeral 40 shows a first step in the invention which is labeled “Determination of the Causative Agent”. A sample of an infected nail can be obtained by several methods. In general, the nail surface and surrounding areas are cleaned with an antiseptic and samples of the infected area are removed by any acceptable method, which acceptable methods include cutting, burring and curetting. The sample obtained is then placed on various media depending on the suspected organism. These medias, for example, can be Sabourads media, a potato media, DTM, as well as other medias. The suspected organism is placed on the media and a culture is grown. Usually, at least two different medias are required, as any two of the above can be employed. The causative organism is then identified by a combination of various standard clinical tests. This is depicted by module 41 of FIG. 4. There is a test which is referred to as potassium hydroxide microscopy or KOH microscopy. In this test, part of the sample is treated with solution of potassium hydroxide and then examined under a low powered microscope. The features are observed and compared to a standard classification system. Once a culture is grown, it is identified by its gross color and texture and through standard bacteriological microscopic examination. This takes into consideration various hyphae structures and cell configurations. By combining the information in steps 40 and 41 as indicated above, the above organism is identified according to traditional classification systems. The most common causative organisms infecting nails are dermatophytes, saprophytes and yeasts. Research has indicated that there are a large variety of micro-organisms infecting nails, which have responded to the organism specific method of treatment and protocol noted here. The following is a partial list of organisms treatable by the approaches as indicated in this specification:

| | |
|---|---|
| SAPROPHYTES | PITHOMYCES |
| | (DERMATACIOUS FUNGUS) |
| MICROSPORUM CANIS | T RUBRUM |
| MICROSPORUM GYPSEUM | T MENTAGROPHYTES |
| CANDIDA ALBICANS | T TONSORANS |
| EPIDERMOPHITE FLOCCOSUM | PENICILLIUM |
| CANDIIDE PARAPSILOSOS | C, GURILLIERMODE |
| C. PARAPSILOSIS | |

Reference numeral 42 refers to the next step of the procedure, where one now selects a medication according to the identified organism. As indicated, once an organism is identified, a chemical medication known to be effective against the organism is chosen. An ionized preparation of the chemical or medication is then prepared 43 in the following way. Some form of the chemical/medication is obtained, which is usually a powder, a pill or a capsule. Once the form of the medication is obtained, the chemical/medication is extracted. For example, when a pill form of a water soluble medication is used, one method of obtaining an ionized solution of the chemical/medication is to triturate the pill into a fine powder. This powder can be, for example, less than 80 mesh. The powder is then added to sterile water to achieve the desired concentration, which is usually a one percent solution. The resulting suspension is continually stirred for six hours to completely dissolve the medication. This is usually done at 25° C. The resulting suspension is then vacuum filtered twice through a 0.22 micron Durapore membrane filter, type GV, to remove inactive, insoluble excipients. If desired, quality assurance can be ascertained by a concentration determination that determines the solution's potency. The solution can then be sent out to an independent company to verify the results. The area to be treated, as for example, the nail in FIG. 1 is then sterilized with the use of an antiseptic so that the portion of the toe or finger that houses the nail and the nail matrix is sterile. This step is shown in Block 44.

Once the area is sterilized, one of the many available commercial moist pad type electrodes is selected with a size corresponding to the region to be treated. This moist type pad electrode is available commercially and has been used, for example, in a number of applications, including EKG and so on. The electrode pad as shown, for example, in FIG. 1 as pad 22 is saturated with one and a half (1.5) to two (2) cc's of the ionized solution. A pad/electrode is selected or prepared with the following qualities:

1. Protects the skin against bums and excessive irritation.

2. Completes the electrical circuitry.

3. Houses the medication to be driven into the diseased areas.

This is depicted by step 45 of FIG. 4 where the pad is saturated with medication. The pad, such as electrode 22, is anchored to the nail and the area over its associated matrix 46. Most electrode pads are self-adhering, but their contact with the body usually has to be reinforced by a non-allergenic adhesive tape. This step is important, as it minimizes any possible shocks or burning that the patient might experience. The ionized drug is administered through an electrode receiving-the same charge as the drug. As indicated, the preferred current, which is supplied by the generator 21, is typically less than five milliamps. This can be DC or a pulsed current. In order to obtain a pulse source, one can utilize a conventional device which is coupled to a battery. Such a device is a multivibrator or other electronic circuit capable of transforming the DC potential into a series of pulses. The pulse width can be varied and selected, as well as the pulse amplitude. In any event, one will readily appreciate that either a fixed DC current or a pulsed DC current can be obtained.

The return electrode is shown by electrode 20 of FIG. 1 and is opposite in charge to the drug and is placed on a neutral side on the body surface. For example, for the treatment of diseased nails of the feet, the return electrode 20 (FIG. 1) is placed at the lower third of the leg being treated. For each electrode used to administer the drug, a return electrode must be used. For example, if the right first toenail and the right fifth toenail are being treated by two separately attached small electrode pads, two return pads should be placed on the right calf. There is essentially one return path for each treated pad. As shown in FIG. 1, there is a timer 24.

There are several timer controlled iontophoresis devices available that adequately and safely drive chemicals/medications into the diseased areas. An appropriate positive or negative electrode lead is attached to the electrode pad containing the medication and the ground electrode. For example, if a drug like Terbinefine is chosen as the drug of choice, positive electrodes would be attached to the electrode pad 22 containing the medication, while the negative electrode 20 would be attached to the ground electrode pad. If a drug like vinegar is chosen, then the negative battery electrode would be attached to the electrode pad 22 containing the medication, while the positive electrode would be attached to the ground electrode pad 20.

As indicated, preferably the battery operated device or generator 21 should have a timing device 24 associated with and be capable of delivering small controlled electrical currents typically in a range of 0.5 to 5 milliamps per minute. This can be achieved in various combinations of power, as for example, 2 milliamps current for 30 minutes. The electrodes are placed over the medicated pads, and are in turn placed over the diseased area, are therefore positioned and controlled accordingly. Once the proper terminal has been determined according to the polarity of the medication being delivered, the treatment is started. The current on the device 21 is slowly raised until the patient can feel a very gentle tingling. This establishes the pain threshold of the patient. The current is then lowered just below this pain threshold level. This is depicted in step 47 of FIG. 4. It is applied for 60 milliamp minutes. For example, if the final current arrived at is 2 milliamps, then the patient is treated for 30 minutes. As one can ascertain, 2 times 30 equals 60, which is 60 milliamp minutes. The patient is treated 48 once a week and can be treated for four weeks and then every other week for an additional four treatments, as depicted in step 48. It has been determined that eight treatments can be extremely beneficial. This protocol as described above has been extremely effective in improving and/or curing the diseased nail plate and matrix. For prophylaxis, the drug of choice is administered every three to six months of the first year and then once a year.

The method and protocol described above can achieve markedly better cure rates, improvement and/or prophylaxis in the treatment of Onychomycosis than the current oral administration of drugs. This technique removes any systemic side effects and at most only minimal local side effects. A main function of the method is to enable one to bypass the systemic route used by oral medication by placing the medication exactly where it is needed. The method is extremely effective, including the treatment and the protocol and is unique in treating diseases of the nail.

As one can ascertain, this invention involves infections of the nail. The nail is a non-vital, dead organic structure capable of housing serious micro-organisms that can lead to amputation of a limb and in compromised patients, death. Normal iontophoresis causes a transference of ions into the microcirculation at the dermo/epidermal junction. This protocol transfers the ions directly into the nail matrix and nail plate where there is very little circulation. Therefore, one only has to deliver a small amount of medication to the diseased areas. The medication is absorbed at a very slow rate and therefore maintains its effectiveness over a long period of time. Based on research, it is determined that the medication can remain effective for a week or more, which accounts for the initial treatments being applied at one week intervals. Once the invading micro-organism is destroyed, the treatment period can be spaced at further intervals. The research supports the above-noted method and protocol.

Basically, the concept of iontophoresis is a relatively simple principle. The principle is that similarly charged ions repel each other. Therefore, to drive the positive sodium ion in a salt solution into a tissue, one would apply a positive charge to it. Similarly, to drive terbinefine a positively charged ion into the nail and nail matrix one places an ionized solution of terbinefine under the positive electrode. Modern iontophoresis is operated with a 9-volt battery, which has enough power to drive ions through the tissue, but is not strong enough to create a burn. Special pads that protect the skin and can hold ionized solutions are employed and commercially available. The concept of iontophoresis is extremely old and was first described in 1747 by Veratti. It is also understood that iontophoresis was employed in the past, especially in the 1930's for the transport of substances into the skin. According to research and as far as the present applicants are concerned, it is not known to them than anyone has ever utilized this technique for curing or treating various infections of the nail matrix and nail plate. It is totally unobvious to do this, as the conventional and most effective methods are associated with extreme side effects, which can be extremely detrimental to a healthy individual's immune system, as well as to the individual's health in general.

While certain embodiments have been shown in detail, it will be apparent to those skilled in the art that many alternatives may be employed and these are deemed to be included within the spirit and scope of the appended claims.

What is claimed is:

1. A method for the treatment of infections of the nail matrix and nail plate of a mammal, comprising the steps of:
placing at least a first electrode on the infected nail and overlying said nail matrix and nail plate,
saturating said electrode with a medication effective against said infection,
placing at least a second electrode on the mammal near to said nail matrix and plate,
causing a current to flow through said first and second electrodes to deliver said medication to the nail plate and nail matrix of said mammal.

2. The method according to claim 1 wherein said first electrode is of opposite polarity to said second electrode.

3. The method according to claim 1 wherein said infection is Onychomycosis and said medication is Lamisil.

4. The method according to claim 1 wherein said step of causing a current to flow includes the step of connecting said first electrode to one terminal of a current generator and connecting said second terminal to said other terminal of said current generator.

5. The method according to claim 1 wherein said current generator is a battery.

6. An apparatus for the treatment of infections of the nail matrix and nail plate of a mammal comprising:
a transdermal molecular device having first and second electrodes, each electrode associated with a pad to enable one electrode to be placed over the infected nail matrix and nail plate and said electrode having an absorbent area adapted to receive a medication effective to treat said infections and said second electrode adapted to be placed on a body portion of said mammal in proximity to said infected nail matrix and plate.

7. The apparatus according to claim 6 further including a selectively adjustable timer connected to said device to cause a current to flow through said first and second electrodes during a predetermined time period.

8. A method for the treatment of various infections of the nail matrix and nail plate of mammals comprising the steps of:
determining the causative agent causing the infection,
selecting a medication known to be effective in treating said determined causative agent,
preparing an ionized solution of said medication,
saturating a first electrode pad with said ionized solution for placing said saturated first electrode pad over the infected nail matrix and nail plate area of said mammal,
placing a second electrode on said mammal near said first electrode pad,
causing a current to flow through said first and second electrodes for a predetermined period effective to deliver said medication to said nail matrix and nail plate of said mammal.

9. The method according to claim 8 wherein said predetermined time is between 20 to 40 minutes.

10. The method according to claim 9 wherein said current flow is between 0.5 to 5 milliamps.

11. The method according to claim 8 wherein said steps of causing a current flow is accomplished by connecting said first electrode to a first terminal of a current generator and said second electrode to a second terminal of said current generator.

12. The method according to claim 11 wherein said current generator is a battery.

13. The method according to claim 11 wherein said current generator is a pulsed DC current generator.

14. The method according to claim 8 including the further step of determining the current level to be applied to said mammal by varying the current between the electrodes until the mammal feels a tingle and then lowering the current just below the tingle current level.

15. A method for the treatment of various infections of the nail matrix and nail plate of a mammal, comprising the steps of:
placing a first electrode having an absorbent pad over the infected nail matrix and nail plate,
placing a second electrode on the body of said mammal in proximity to said first electrode,
saturating said absorbent pad of said first electrode with an ionized solution of a medication known to be effective in the treatment of said infected nail,
causing a current to flow between said first and second electrodes for a period sufficient to cause said ionized solution to be delivered directly into the infected areas of said nail plate and nail matrix.

16. The method according to claim 15 wherein said current flow is between 0.5 to 5 milliamps.

17. The method according to claim 15 wherein said current is caused to flow for a period between 15 to 60 minutes.

18. The method according to claim 15 wherein said mammal is a human.

19. The method according to claim 18 wherein said nail plate and nail matrix are finger or toenail plates or nail matrixes.

20. The method according to claim 15 wherein said infection is Onychomycosis.
